(19) United States
(12) Patent Application Publication
(10) Pub. No.: US 2005/0049331 A1
(43) Pub. Date: Mar. 3, 2005

(54) Microporous latex membranes, related articles and methods

(75) Inventors: Joaquim Coutinho, Ribeirao Preto (BR); Fatima Mrue, Goiania (BR)

Correspondence Address: SIDLEY AUSTIN BROWN & WOOD LLP, 1501 K STREET, N.W., WASHINGTON, DC, 20005, US

(73) Assignee: Pele Nova Biotecnologia S.A., Sao Paulo (BR)

(21) Appl. No.: 10/892,442

(22) Filed: Jul. 16, 2004

Related U.S. Application Data
(60) Provisional application No. 60/488,008, filed on Jul. 18, 2003.

(51) Int. Cl. A61F 13/00

(52) U.S. Cl. 523/307

(57) ABSTRACT

Novel microporous latex membranes having advantageous properties are disclosed as are methods for making and using the microporous latex membranes. Disclosed processes for making such membranes include those processes comprising applying a latex suspension to a surface to result in a latex coated surface and subjecting the latex coated surface to a first temperature less than about −2° C., and subsequent thereto, heating the latex coated surface at a second temperature to result in a microporous latex membrane. Moreover, it is disclosed that certain properties of the microporous membranes may be manipulated through judicious selection of first and second temperatures.

### CROSS-REFERENCE TO RELATED APPLICATION

This application claims the benefit of U.S. Provisional Application No. 60/488,008, filed Jul. 18, 2003.

### BACKGROUND OF THE INVENTION

1. Field of the Invention

The invention relates to microporous latex membranes and to methods of making and using such membranes. In particular applications, the invention relates to those microporous latex membranes suitable for use in clinical applications including wound healing.

2. Description of Related Art

Angiogenesis is the development of new blood vessels from pre-existing arteries and veins and has become an area of increased interest in both scientific and clinical application purposes. Therapies directed towards stimulating the angiogenic process seek to accelerate the development of new blood vessels in tissues with an insufficient microvascular supply. Wound healing cannot occur without angiogenesis as the vasculature mainly comprises the repair tissue, normally denominated granulation tissue. (See, for example, Singer et al., N. Engl. J. Med. 341, 738-746 (1999); Harding, et al., BMJ 324, 160-163 (2002); and Pierce et al., Ann. Rev. Med. 46, 467-481 (1995), the entireties of which are each herein incorporated by reference.)

Clinically, the largest categories of nonhealing wounds are classified as chronic cutaneous ulcers, as diabetic and venous foot ulcers, pressure sores, burns and ulcers of other etiologies. Traditionally, the treatment of chronic nonhealing wounds has been dominated by dressings that primarily function to support the good conditions for the natural course of wound healing, such as to maintain a moist wound healing environment; to absorb drainage; to debride the wounds; to cushion and protect the wound from trauma and gross contamination; to support, splint or immobilize the body part and incisional area; to aid the hemostasis and minimize edema as in a pressure dressing; to enhance the patient physical comfort and aesthetic appearance; to prevent cell dehydration; and to apply medications.

Recent advances in the therapy of chronic wounds have involved the use of growth factors that act directly in one of the steps of the healing, such as the formation of new capillaries (angiogenesis) accelerating the rate of formation of the granulation tissue and epithelialization. In this context, platelet derived growth factor (PDGF), which is manufactured by Ortho McNeil—USA as Regranex® (becaplermin), has been prescribed for the treatment of diabetic foot ulcers.

A variety of skin derivatives are also available where acute or chronic wounds are covered by those derivatives. Those are autologous or allogeneic grafts of dermal components or epidermal cells with or without dermal components. The disadvantages of the use of those grafts include: the time needed to grow enough tissue (epidermal, dermal or both of them), biopsies, the high cost of the materials and the needed surgical procedures to implant the cultured grafts.

WO9815300A1, the entirety of which is herein incorporated by reference, discloses essentially non-porous latex membranes and teaches their suitability for use in substitution, reconstruction or regeneration of human or animal organs or tissues. The disclosed non-porous latex membranes are taught to comprise the polymerization product of natural latex as its main constituent.

Although various dressings and treatments are known for treatment of wounds, all or almost all of the known dressings and treatments suffer from one or more disadvantages. Therefore, a need remains for improved dressings and treatments for wounds.

### SUMMARY OF THE INVENTION

An aspect of the invention resides in the discovery that novel microporous latex membranes having advantageous properties may be obtained through a process comprising applying a latex suspension to a surface to result in a latex coated surface and subjecting the latex coated surface to a first temperature less than about −2° C., and subsequent thereto, heating the latex coated surface at a second temperature to result in microporous latex membrane. Moreover, another aspect of the invention resides in the discovery that certain properties of the microporous membranes may be manipulated through judicious selection of first and second temperatures. For example, where the first temperature is a temperature within the range from about −2° C. to about −80° C., the resulting membrane exhibits permeability to molecules including oxygen, carbon dioxide, water vapor, Evans blue, bovine albumin and immunoglobulin G, but is essentially impermeable to blue dextran. While not wishing to be bound by theory, Applicant believes that the formation of water crystals upon cooling underlies the microporous nature of the membranes resulting from the method.

The invention is also directed to methods using various embodiments of the microporous membranes of the instant invention, including use in clinical applications such as wound healing.

Given the abundant availability and other advantages of natural latex, an object of this invention is to provide microporous latex membranes having special microarchitecture. The invention achieves this object. Other objects, features and advantages of the present invention will be more readily apparent from the detailed description of the preferred embodiments set forth below taken in conjunction with the accompanying drawings.

### BRIEF DESCRIPTION OF THE DRAWINGS

FIG. 1 depicts a scanning electron micrograph (SEM) of the surface of a non-porous latex membrane of the type disclosed in WO 98/15300 A1.

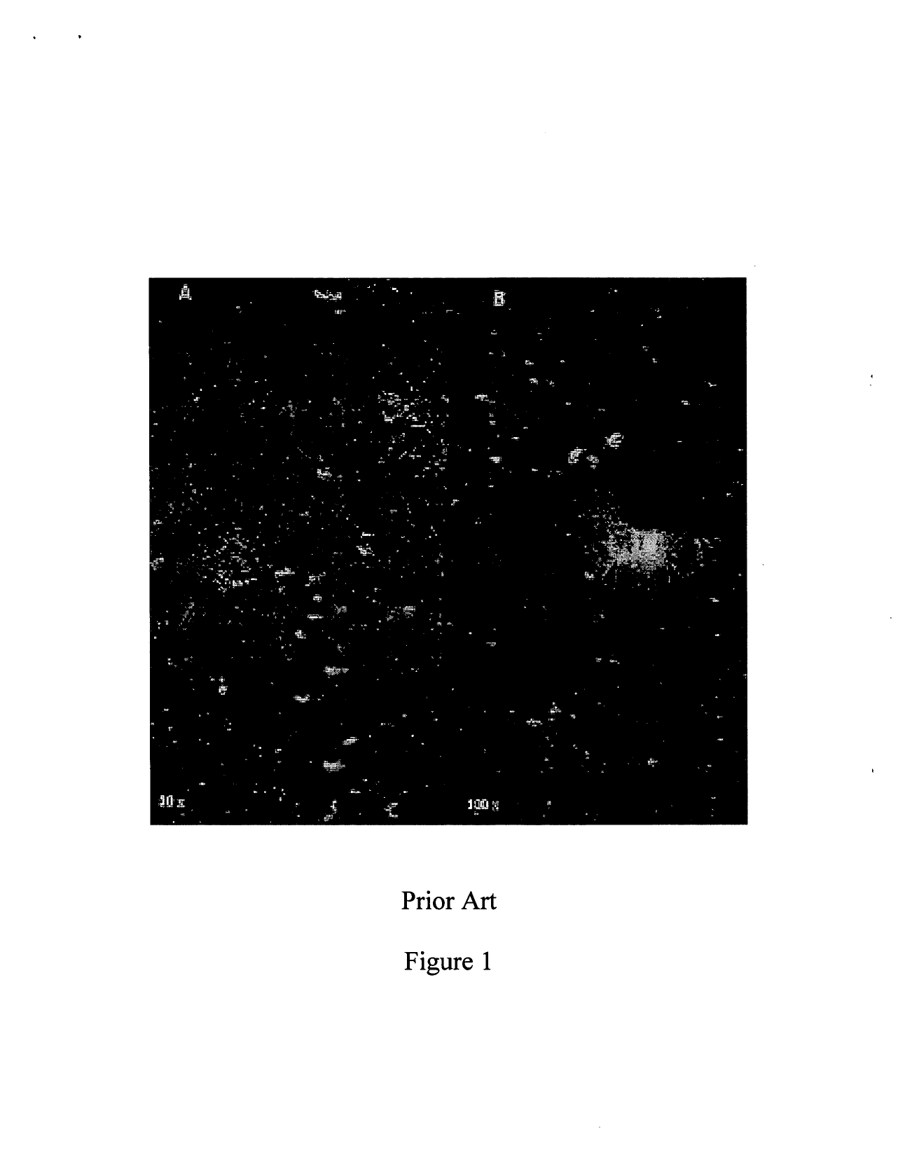

FIG. 2 depicts a SEM of the surface of a preferred microporous latex membrane of the instant invention.

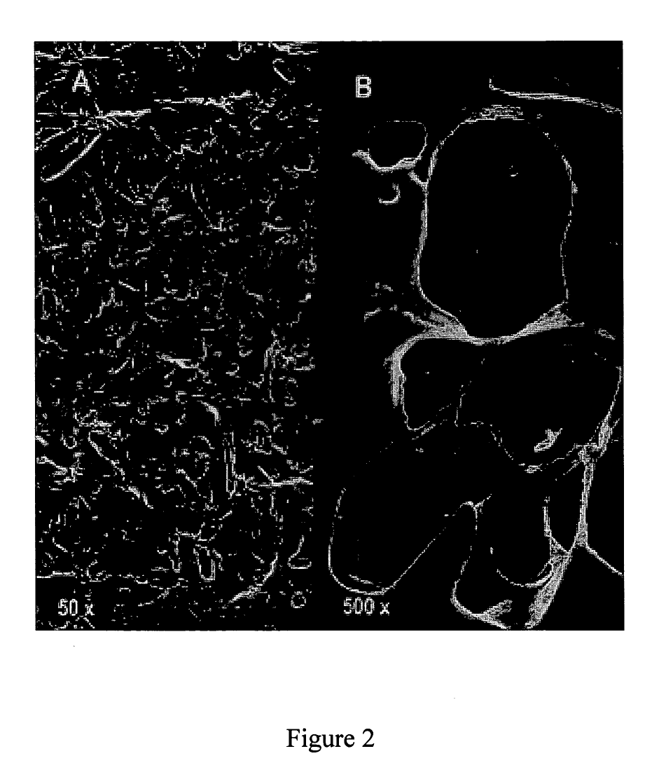

### DETAILED DESCRIPTION OF THE PREFERRED EMBODIMENTS

The instant invention contemplates a variety of embodiments. A preferred embodiment of the instant invention relates to clinical uses as well as to methods to prepare microporous latex membranes. Given that natural latex harvested from plants including, for example, Hevea brasiliensis, Hancornia speciosa Gomez (mangabeira), Couma macrocarpa (sorva), Ficus indica, Parthenium argentatum (guayule) and subspecies thereof, is purported to contain an angiogenic and/or chemoattractant protein, especially preferred embodiments make use of these sources of latex. Among such especially preferred embodiments are those microporous latex membranes that are prepared using processes that maintain angiogenic and/or chemoattractant activity during the polymerization process. Preferred embodiments of such processes include those in which the temperature during polymerization does not exceed about 70° C., above which temperature the angiogenic and/or chemoattractant activities are diminished. Although not wishing to be bound by theory, applicants believe a 35,000 Dalton protein is responsible for the observed angiogenic and/or chemoattractant activities, and postulate that this protein loses function at temperatures above 70° C.

In preferred embodiments, when that biomembrane is implanted or placed directly over the wound, as a dressing, or is used as a molded prosthesis, (1) the special microporous microarchitecture obtained during the polymerization and (2) the angiogenic and chemoattractant factors that are trapped in the polymer and then are released induce angiogenesis and attract monocytes and other inflammatory cells to the local of the wound. These monocytes/macrophages attracted and adhered to the surface of that microporous biopolymer are stimulated to release mediators such as chemotactic and growth factors that will act synergistically with the natural angiogenic and chemoattractant factors of the natural latex.

In preferred embodiments of the invention, a goal is the stimulation of the angiogenic response in wounds, around wounds and/or within porous of films or prosthesis implanted of that material, such as vascular grafts, special meshes, biohybrid organs, immunoisolating capsules as pancreatic islets for treatment of diabetic patients. When that microporous biomembrane is implanted in animals, they showed an optimal biocompatibility, and around the biomembrane appears a thin fibrous capsule very well neovascularized.

The microporosity on the surface of the biomembrane stimulates: the adherence of macrophages, the formation of a fibrous capsule without the presence of foreign body giant cells, and permanent neovascularization mainly in the interface tissue-biomembrane. The special microarchitecture of that polymer stimulates elicited (resident) macrophages which adhere to the polymer surface to sustain the release of growth factors, such as the vascular endothelial growth factor (VEGF) which induces and maintains the high number of neovessels in the interface polymer-tissue of the fibrous capsule, by way of non-limiting examples.

Either in the format of a film or as other devices manufactured in the same way, the biomembrane may be used in various situations where angiogenesis and wound repair promotion is the goal. As a dressing, the biomembrane may be used for the treatment of chronic cutaneous ulcers, mainly diabetic neurophatic ulcers, pressure sores, venous and chronic ulcers of other etiologies and burns. As an implanted device, it may be used as a biohybrid organ for macroencapsulation of cells in situations where immunoisolation is desired. It may be used to deliver chemotherapeutic drugs, hormones or other drugs in situations where long-term and regular administration is needed. As a prosthesis, it may be used as a substitute for organs as arteries and veins, testicles, eyes, meshes for abdominal hernia repair, facial prosthesis for resurfacing the skin. Polymers grafted with the biomembrane, where they are used as a skeleton for the biomembrane, have the same application.

Among preferred uses for the disclosed microporous latex membranes are those involving the use of the membrane where it is grafted to natural, synthetic or semi-synthetic polymers. The microporous latex membranes of the instant invention, including those employed as films as well as those grafted to other polymers, are useful to induce angiogenesis, to accelerate the formation of granulation tissue and wound repair in nonhealing chronic ulcers and burns. They also find utility in methods to manufacture/prepare wearing cloths for burns; dressings; implanted devices to deliver drugs (such as chemotherapeutic, hormones and antibiotics); devices to the macroencapsulation of cells, biohybrid organs; prosthesis for arteries and veins; prosthesis for the resurfacing of the skin; meshes for abdominal hernia repairing and peritonostomy; and prosthesis for organs such as testicles and ocular. Moreover, the microporous latex membranes of the instant invention, including those employed as films as well as those grafted to other polymers, may be used in guided bone regeneration by means of functioning as a barrier membrane as well as in endochondral bone formation as inductor of angiogenesis of the growth plate, regeneration of the periodontal tissue and accelerating the osteo (bone) formation around dental or orthopedics implants.

### EXAMPLE 1

Natural latex milked (harvested) from the rubber tree Hevea brasiliensis was treated with 7% of ammonia (final concentration, v/v), centrifuged to obtain a density of 0.948 and then filtered. After the adjustment of the density to the desired content of rubber (30-60% of rubber) with pure water, then a suspension of sulfur at 50%, 10-40 grams per liter of latex were added with slow agitation. Then, 20 ml of a 10% solution of polyvinyl-ethyl-ether by kg of latex (w/v) were added through slow agitation. That suspension was stored at 4° C. until used to prepare films of the biomembrane or others devices. The molding of the resulting microporous membrane was accomplished by spraying the natural latex suspension onto a smooth and flat surface, and immediately transferring to a freezer having a temperatures in the range from −2° to −80° C. That suspension rested until it became frozen and polymerized (generally about 30 minutes to 2 hours). The solidified material was transferred to an oven (hood) at 70° C. to complete the polymerization and dry for 30 minutes. The film (biomembrane) was wrapped and sterilized either under ethylene oxide or irradiation (gamma ray).

### EXAMPLE 2

A prosthesis coated with a microporous latex membrane of the invention was manufactured by dipping a mold for a prosthesis in a suspension of latex, followed by freezing and polymerization steps analogous to those described in Example 1. The beneficial properties of the film obtained under this procedures, maintaining the activities of the angiogenic and chemoattracting factors trapped in the complacent and microporous biomembrane, could be transferred when used to graft natural, synthetic or semi-synthetic polymers as polyurethanes, polyamides, polyesters, celulloses. Such is achieved when the polymers are dipped in the natural latex suspension and the polymerization takes place under low temperatures (−2° to −80° C.), following the procedures described.

While a number of embodiments of this invention have been herein before described, it is apparent that the basic embodiments can be altered to provide other embodiments of the disclosed invention. Therefore, it will be appreciated that the scope of this invention includes all alternative embodiments and variations which are described in the foregoing specification and by the claims appended hereto. The invention is not to be limited by the specific embodiments that have been presented herein by way of example.

1. A microporous latex membrane obtainable by a process comprising:
(1) applying a latex suspension to a surface to result in a latex coated surface;
(2) subjecting the latex coated surface to a first temperature in a range from about −2° C. to about −80° C.; and subsequent thereto,
(3) heating the latex coated surface at a second temperature in a range from about 25° C. to about 70° C. to result in microporous latex membrane.

2. The microporous latex membrane of claim 1 having angiogenic and chemoattracting activities.

3. The microporous latex membrane of claim 1, wherein the latex suspension comprises latex extracted from one or more rubber trees selected from Hevea brasiliensis, Hancornia speciosa, Couma macrocarpa, Ficus indica, Parthenium argentatum, or subspecies thereof.

4. The microporous latex membrane of claim 3, wherein the latex suspension comprises latex extracted from one or more rubber trees selected from Hevea brasiliensis or subspecies thereof.

5. The microporous latex membrane of claim 1, wherein the step of applying the latex suspension to the surface comprises spraying the latex suspension onto the surface.

6. The microporous latex membrane of claim 1, wherein the latex membrane is permeable to molecules including oxygen, carbon dioxide, water vapor, Evans blue, bovine albumin and immunoglobulin G, but is essentially impermeable to blue dextran.

7. A method for preparing a microporous latex membrane, the method comprising the following steps:
(1) applying a latex suspension to a surface to result in a latex coated surface;
(2) subjecting the latex coated surface to a first temperature of about −2° C. or lower; and subsequent thereto,
(3) heating the latex coated surface at a second temperature to result in microporous latex membrane.

8. The method of claim 7, wherein the first temperature is in a range from about −2° C. to about −100° C.

9. The method of claim 8, wherein the first temperature is in a range from about −2° C. to about −80° C.

10. The method of claim 9, wherein the first temperature is in a range from about −2° C. to about −20° C.

11. The method of claim 9, wherein the first temperature is in a range from about −20° C. to about −40° C.

12. The method of claim 9, wherein the first temperature is in a range from about −40° C. to about −60° C.

13. The method of claim 9, wherein the first temperature is in a range from about −40° C. to about −60° C.

14. The method of claim 9, wherein the first temperature is in a range from about −60° C. to about −80° C.

15. The method of claim 8, wherein the first temperature is in a range from about −80° C. to about −100° C.

16. The method of claim 7, wherein the first temperature is about −100° C. or lower.

17. The method of claim 7, wherein the second temperature is above about 25° C.

18. The method of claim 7, wherein the second temperature is below about 70° C.

19. The method of claim 7, wherein the second temperature is in a range from about 25° C. to about 70°C.

20. The method of claim 7, wherein the first temperature is in a range from about −2° C. to about −80° C. and the second temperature is in a range from about 25° C. to about 70° C.

21. The method of claim 7, wherein the step of applying the latex suspension to the surface comprises spraying the latex suspension onto the surface.

22. The method of claim 7, wherein the first temperature is in a range from about −2° C. to about −80° C., the second temperature is in a range from about 25° C. to about 70° C., and the step of applying the latex suspension to the surface comprises spraying the latex suspension onto the surface

23. A microporous latex membrane obtainable by the method of any one of claims 8-22.

24. A microporous latex membrane obtained by the method of claim 23.

25. A microporous latex membrane having permeability for molecules including oxygen, carbon dioxide and water vapor, wherein the latex membrane is essentially impermeable to blue dextran.

26. The microporous latex membrane of claim 25, wherein the latex membrane is essentially impermeable to blue dextran and immunoglobulin G.

27. The microporous latex membrane of claim 26, wherein the latex membrane is essentially impermeable to blue dextran, immunoglobulin G and bovine albumin.

28. The microporous latex membrane of claim 27, wherein the latex membrane is essentially impermeable to blue dextran, immunoglobulin G, bovine albumin and Evans blue.

29. The microporous latex membrane of claim 25, wherein the latex membrane is permeable to molecules including oxygen, carbon dioxide, water vapor and Evans blue.

30. The microporous latex membrane of claim 29, wherein the latex membrane is permeable to molecules including oxygen, carbon dioxide, water vapor, Evans blue and bovine albumin.

31. The microporous latex membrane of claim 30, wherein the latex membrane is permeable to molecules including oxygen, carbon dioxide, water vapor, Evans blue, bovine albumin and immunoglobulin G.

32. The microporous latex membrane of claim 25 having angiogenic and chemoattracting activities.

33. The microporous latex membrane of claim 25, wherein the latex membrane comprises latex extracted from one or more rubber trees selected from Hevea brasiliensis, Hancornia speciosa, Couma macrocarpa, Ficus indica, Parthenium argentatum, or subspecies thereof.
